(19) United States
(12) Patent Application Publication
(10) Pub. No.: US 2025/0204896 A1
(43) Pub. Date: Jun. 26, 2025

(54) ASSEMBLY AND METHOD FOR SPLIT SALIVA COLLECTION AND TESTING

(71) Applicant: Premier Biotech, LLC, Minneapolis, MN (US)

(72) Inventors: Larry R. MASTERSON, Minneapolis, MN (US); Jennifer M. Colby, Minneapolis, MN (US); Steve Ekbom, Minneapolis, MN (US); Todd Bailey, Bonita Springs, FL (US)

(21) Appl. No.: 18/990,081

(22) Filed: Dec. 20, 2024

Related U.S. Application Data
(60) Provisional application No. 63/613,558, filed on Dec. 21, 2023.

(51) Int. Cl. A61B 10/00

(52) U.S. Cl. CPC A61B 10/0051

(57) ABSTRACT

An oral fluid collection assembly comprises a collector having a stem carrying a collection media at a distal end thereof. A transfer container has an extraction surface separating an interior of the transfer container into a first volume and a second volume. The transfer container and the collector are connectable by latching features that cause engagement of the collection media and the extraction surface to produce an extracted sample. The transfer container has an outlet at its distal end so that the extracted sample can be dispensed from the transfer container. Preferably, a plurality of laboratory containers configured to receive portions of the extracted sample from the transfer container are also provided.

### CROSS-REFERENCE TO A RELATED APPLICATION

This application is based upon and claims priority to provisional application Ser. No. 63/613,558, filed Dec. 21, 2023, which is incorporated fully herein by reference for all purposes.

### FIELD OF THE INVENTION

The present invention relates to devices and methods for collecting and testing saliva. Embodiments of the present invention relate to saliva collection and stabilization of the sample prior to testing for one or more substances.

### BACKGROUND OF THE INVENTION

Bodily fluids are collected for a variety of applications that may include, but are not limited to, diagnosis for illness, determining the presence of illicit substances, confirming or establishing levels of therapeutic agents, and RNA/DNA profiling. For such purposes, the types of bodily fluids collected include blood, urine, hair, and saliva. Saliva often represents compounds that are currently in the system, similar to blood, but the collection of saliva is minimally invasive as compared to blood collection. Privacy concerns make direct observation of saliva collection more facile as opposed to urine collection. This makes saliva advantageous in situations such as drugs of abuse testing and RNA/DNA testing. Last, the analysis of hair is typically associated with the presence of compounds that were present in an individual weeks or months prior, which limits the utility in the detection of illicit substances that may be life threatening or for situations such as impaired driving under the influence of drugs.

Split specimens are often used in laboratory testing where one sample is used for testing at one location or time, while the other is preserved for future analysis or used for testing at another location or time to confirm reproducibility or for retesting needs. Split specimens should represent identical samples. Identical samples are the same sample type (blood, urine, hair, or saliva) taken at the same time from an individual. Split specimens for urine and blood can be quickly accommodated by directly dividing a large aliquot of sample collected. For urine and blood, this is easily done since these specimens possess fluidity that resembles water and can be split. Two specimens may also be collected for blood by simply changing the vessel used during collection. Further, the volume of these specimens is often tens of milliliters (mL), which makes handling of the sample at the point of collection much easier when the sample needs to be divided. So long as sufficient hair specimen is available, dividing hair specimens can be done by mass after collection or by taking two samples near the same time. Saliva poses very different challenges when it comes to split sample collection. This is due to the lower volume of collection (between 1-3 mL) and the inherent viscosity of saliva which makes handling it much more difficult at the point of collection.

The present invention recognizes and addresses considerations of prior art constructions and methods.

### SUMMARY OF THE INVENTION

Embodiments of the present invention provide an oral fluid collection assembly, comprising a collector having a stem carrying a collection media at a distal end thereof. A transfer container has an extraction surface separating an interior of the transfer container into a first volume and a second volume. The transfer container and the collector are connectable by latching features that cause engagement of the collection media and the extraction surface to produce an extracted sample. The transfer container has an outlet at its distal end so that the extracted sample can be dispensed from the transfer container. Preferably, a plurality of laboratory containers configured to receive portions of the extracted sample from the transfer container are also provided.

Some exemplary embodiments include an adaptor to which the transfer container and at least two of the laboratory containers are connectable. The adaptor may define therethrough a flow passage from the outlet of the transfer container to respective inlets of the laboratory containers.

Preferably, the collection media may comprise a sponge mounted to a rigid mounting structure at the distal end of the stem. In addition, the collector may comprise a wicking strip providing a visual indication in the presence of the oral fluid. For example, the wicking strip may be internal to the stem and viewable through a transparent window in the stem.

In some exemplary embodiments, the collector may comprise a handling structure at a proximal end thereof. The handling structure may comprise a cap defining inner threads, the inner threads engaging outer threads on the transfer container. In some exemplary embodiments, the extraction surface may comprise a disc situated at a fixed location in the transfer container, the disc defining at least one aperture (e.g., a plurality of apertures such as radial slots) for passage of the oral fluid.

In some exemplary embodiments, the transfer container is squeezable and the outlet of the transfer container comprises a dropper structure.

In some exemplary embodiments, the transfer container has volume indicia to facilitate dispensing portions of the extracted sample.

In some exemplary embodiments, the outlet of the transfer container is formed by a pierceable membrane.

Another aspect of the present invention provides an oral fluid collection assembly, comprising a collector having a stem carrying a rigid mounting structure at a distal end thereof to which a collection sponge is mounted. The collector in this embodiment further has a cap defining inner threads at a proximal end of the stem. A transfer container having an extraction surface separating an interior of the transfer container into a first volume and a second volume is also provided. The extraction surface defines at least one aperture for passage of an extracted sample of the oral fluid. In addition, the transfer container of this embodiment has outer threads at its proximal end which are engageable by the inner threads of the cap in a manner that causes engagement of the collection sponge and the extraction surface. In addition, the transfer container has an outlet at its distal end so that the extracted sample can be dispensed from the transfer container. A plurality of laboratory containers configured to receive portions of the extracted sample from the transfer container are also provided.

A still further aspect of the present invention provides a method of collecting a saliva sample from a user. One step of the method involves obtaining an oral fluid collector having an elongate stem with a collection sponge located at its distal end. Another step of the method involves collecting a sample with the collection sponge. According to another step, it is determined whether the sample is sufficient. If the sample is sufficient, the sponge is compressed so as to produce an extracted sample into a squeezable transfer container having a dropper structure. According to another step, the transfer container is squeezed so as to dispense respective portions of the extracted sample into at least two separate laboratory containers.

The accompanying drawings, which are incorporated in and constitute a part of this specification, illustrate one or more embodiments of the disclosure and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended drawings, in which:

FIG. 1 is a diagrammatic representation of an oral collection kit in accordance with an embodiment of the present invention;

FIG. 2 illustrates use of a collector of the kit of FIG. 1 to collect a saliva sample;

FIG. 3 is an isometric view of the collector and transfer container of the kit of FIG. 1 when connected;

FIG. 4 diagrammatically illustrates use of the transfer container of FIG. 3 to split the sample into multiple laboratory containers;

FIG. 4A diagrammatically illustrates an alternative interface between transfer container and laboratory container;

FIG. 5 shows the laboratory containers of FIG. 4 closed and packaged for transport to the laboratory;

FIGS. 6 and 7 diagrammatically illustrate an alternative arrangement utilizing an adaptor that allows simultaneous transfer of multiple sample portions; and

FIG. 8 is a flow chart illustrating steps according to exemplary methodology of the present invention.

Repeat use of reference characters in the present specification and drawings is intended to represent same or analogous features or elements of the invention according to the disclosure.

### DETAILED DESCRIPTION OF PREFERRED EMBODIMENTS

Reference will now be made in detail to presently preferred embodiments and presently preferred methodology of the invention, one or more examples of which are illustrated in the accompanying drawings. Each example is provided by way of explanation, not limitation, of the invention. In fact, it will be apparent to those skilled in the art that modifications and variations can be made in the present invention without departing from the scope and spirit thereof. For instance, features illustrated or described as part of one embodiment (or method) may be used on another embodiment (or method) to yield a still further embodiment (or method). Thus, it is intended that the present invention covers such modifications and variations as come within the scope of the appended claims and their equivalents.

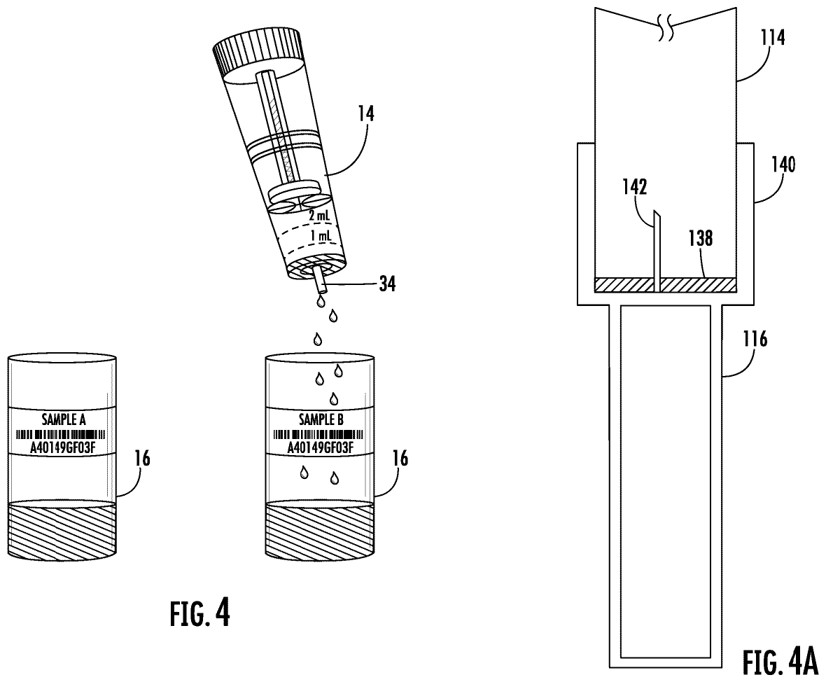

As used herein, terms referring to a direction or a position of the transfer container and/or laboratory containers, such as but not limited to “vertical,” “horizontal,” “top,” “bottom,” “above,” or “below,” refer to directions and relative positions with respect to the orientation generally shown in FIG. 4. Further, the term “or” as used in this document is intended to mean an inclusive “or” rather than an exclusive “or.” That is, unless specified otherwise, or clear from the context, the phrase “X employs A or B” is intended to mean any of the natural inclusive permutations. Therefore, the phrase “X employs A or B” is satisfied by any of the following instances: X employs A; X employs B; or X employs both A and B. In addition, the articles “a,” “an,” and “the” as used in this document should generally be construed to mean “one or more” unless specified otherwise or clear from the context to be directed solely to a singular form. The meaning of “in” may include “in” and “on.” The word “at” may include “at,” “adjacent to,” and “on.” The phrase “in one embodiment,” as used herein does not necessarily refer to the same embodiment, although it may. The meanings identified above do not necessarily limit the terms, but merely provide illustrative examples for the terms.

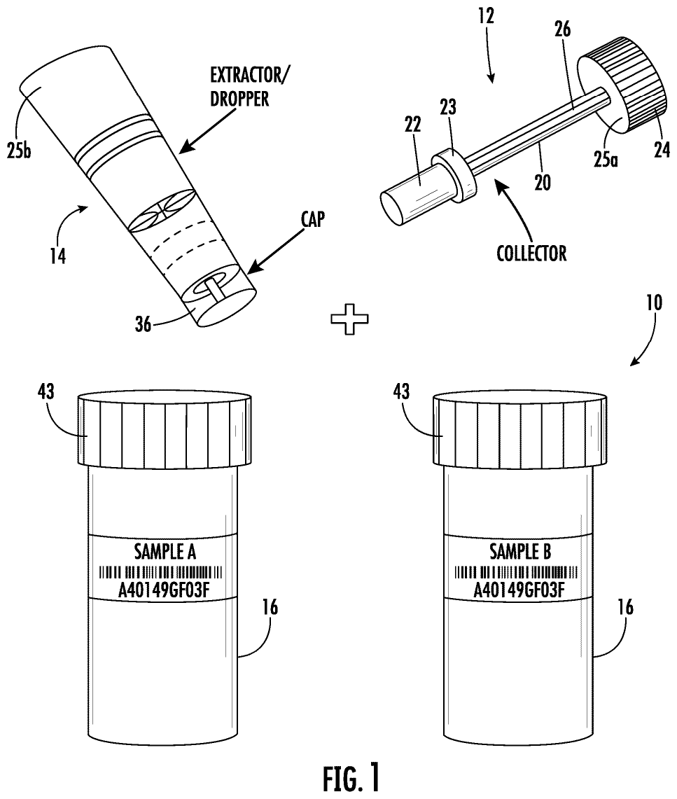

Aspects of the present invention provide a solution for the collection and subsequent splitting of a saliva sample into multiple laboratory containers. As explained below, the laboratory containers may contain storage buffer to preserve the analytes of interest during transport and storage. In this regard, FIG. 1 illustrates an oral fluid collection kit 10 in accordance with an embodiment of the present invention. Kit 10 generally includes a collector 12, a transfer container 14, and a plurality (here, two) laboratory containers 16.

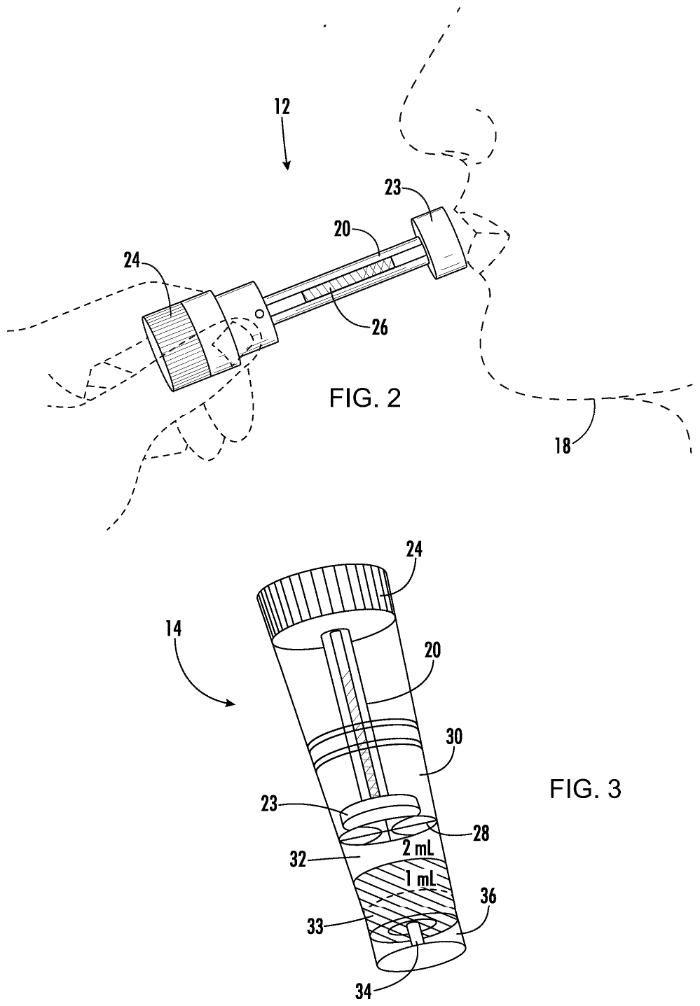

Referring now also to FIG. 2, collector 12 functions to obtain a sample of oral fluid (saliva) from the mouth of a testing subject 18. In this embodiment, collector 12 comprises an elongate stem 20 having an appropriate collection media located at its distal end. In this case, the collection media takes the form of a compressible sponge that is capable of drawing in a sample of the oral fluid. Preferably, a “low sample loss sponge” will be used for this purpose so that a small percentage (e.g., no more than 10%) of the sample volume or its analytes will remain in the sponge after extraction. Sponge 22 may be suitably mounted to a rigid mounting structure 23 (e.g., disc) at the distal end of stem 20.

A handling structure, here in the form of a cap 24, is located at the proximal end of stem 20. As shown in FIG. 2, the collector 12 is held by the person obtaining the sample (e.g., a nurse) by the cap 24 or other suitable handling structure. Preferably, a wicking strip 26 is located along stem 20 with one end in fluid communication with the sponge 22. Wicking strip 26 provides a visual indication (e.g., change of color (see FIG. 2)) in the presence of the oral fluid. When the length of the visual indication reaches a certain point, the person obtaining the sample knows that a sufficient sample has been collected in the sponge 22. The wicking strip may be inside of stem 20 in preferred embodiments but viewable through a transparent window in the stem.

Referring now to FIGS. 1 and 3, collector 12 is attached to the transfer container 14 after the sample has been collected from the test subject. In this regard, cap 24 (or other handling structure) preferably has latching features that causes extraction of the collected sample from sponge 22. For example, cap 24 may have inner threads (at 25a) that engage outer threads (at 25b) at the proximal end of transfer container 14. As cap 24 is thus rotated relative to the transfer container 14, stem 20 moves axially inside of transfer container 14. This causes sponge 20 to compress against an extraction surface 28 located at a fixed position inside of transfer container 14. Extraction surface 28 divides the interior of transfer container 14 into a first volume 30 in which portions of the collector 12 are located and a second volume 32 serving as a reservoir for the extracted sample (indicated at 33).

In this case, extraction surface 28 comprises a fixed disc which causes the sponge 22 to be compressed, thereby expressing the extracted sample of the oral fluid. A plurality of apertures, here shown as a series of radial slots, allow the extracted sample to pass into volume 32. If the transfer container is held generally vertically as shown, gravity will assist flow of the sample fluid into region 32. Preferably, at least volume 32 of transfer container will be transparent so that a user can see that the sample has been extracted from the sponge 22. Moreover, indicia, here in the form of circular markings around the body of transfer container 14, may be provided to indicate the total volume of the extracted sample as well as to facilitate equal division of the sample into multiple laboratory containers 16. For example, markings indicating both 1 mL and 2 mL of sample volume (as shown) will facilitate splitting of a 2 mL total sample into separate 1 mL portions.

Preferably, the distal end of transfer container 14 has a suitable outlet to allow the extracted sample to be easily transferred into the laboratory container(s) 16. Referring now primarily to FIGS. 3 and 4, the distal end of transfer container 14 is configured in this case having a dropper structure 34 (e.g., a small nozzle) in fluid communication with volume 32. The open end of structure 34 is normally closed until it is desired to dispense the samples. For example, a piece of foil may be removably affixed to the end of structure 34 which can be removed to allow fluid flow. In this case, however, a cap 36 seals the end of structure 34 when the cap is in the closed position. The cap 36 may be completely removable, or simply flip open in various embodiments, as necessary or desired.

With the end of structure 34 open, the extracted sample may be easily divided into multiple laboratory containers 16 (see FIG. 4). Preferably, transfer container 14 may be squeezable, i.e., sufficiently flexible so that a user can force out the sample by slightly squeezing transfer container 14. Alternatively, collector 12 may be removed from transfer container 14 and replaced by a cap having a squeezable portion which can similarly force out the extracted sample when squeezed.

FIG. 4A illustrates an alternative embodiment in which the distal end of a transfer container 114 is sealed by a membrane 138. In this embodiment, laboratory container 116 has a cylindrical structure 140 in which the distal end of transfer container 114 is received. Transfer container 116 has a needle 142, defining a fluid flow path along its length, that passes through membrane 138 as transfer container 114 is moved into the cylindrical structure 140. As a result, the sample can pass directly into the laboratory container 116.

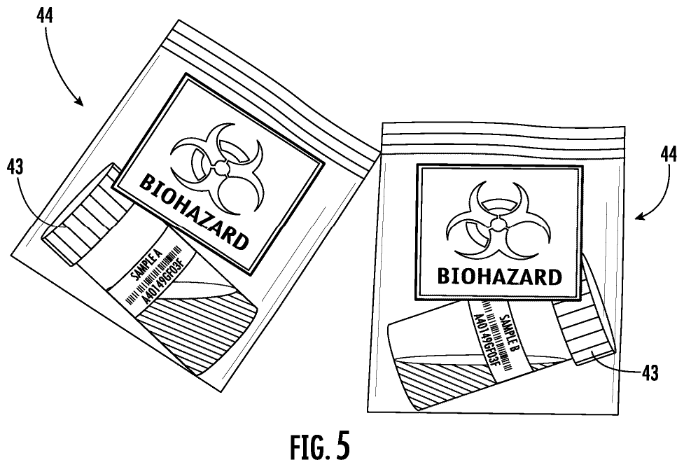

Referring now to FIG. 5, containers 16 (and 116) are referred to herein as “laboratory containers” because they will contain the sample provided to the testing facility to determine whether an analyte of interest is detected. Thus, after the samples are divided into the laboratory containers, they are sealed (e.g., using caps 43) and packaged (as indicated at 44) for storage and/or transport to the testing facility.

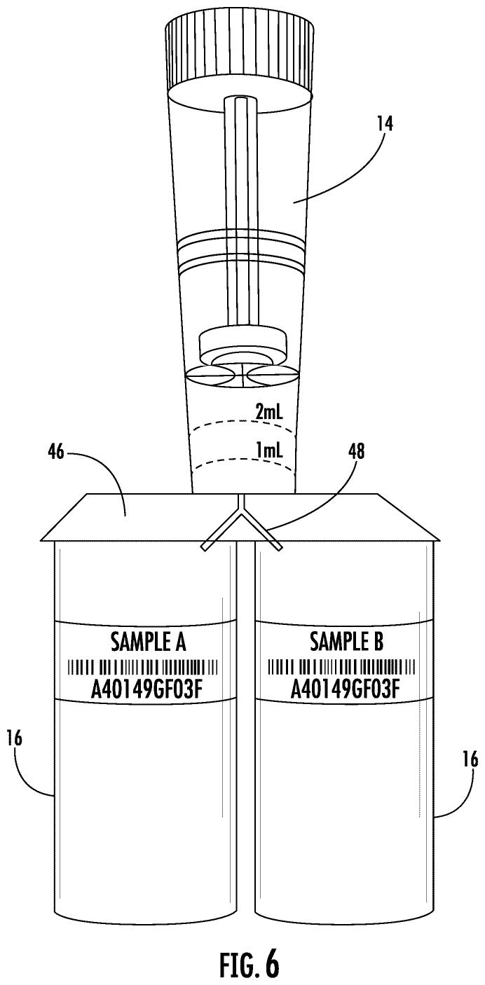

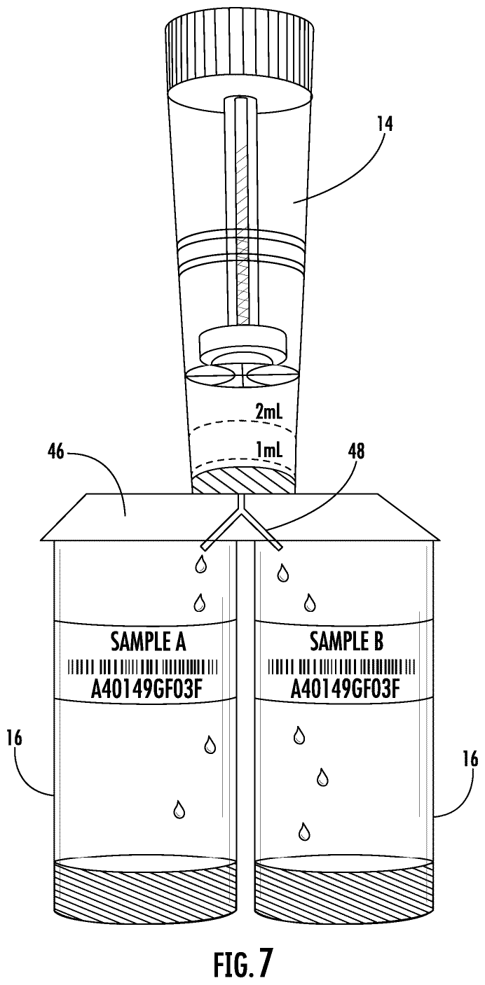

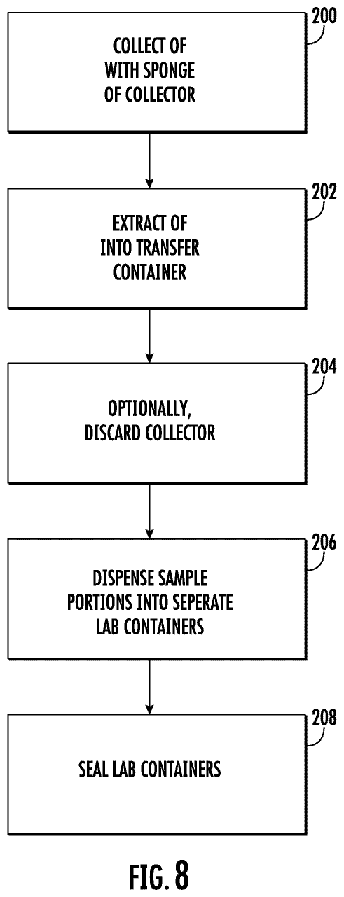

Embodiments are contemplated in which sample portions can be provided to multiple laboratory containers 16 (or 116) simultaneously. In this regard, FIGS. 6 and 7 illustrate the use of an adaptor 46 to which container 14 as well as multiple containers 16 can be connected. Adaptor 46 defines an internal flow path 48 that splits the single outlet of container 14 into respective inlets for the containers 16. Adaptor 46 thus serves as a form of manifold. Containers 16 are then separated from adaptor 46, sealed, and packaged.

In many cases it will be necessary or desirable to provide some sort of additive in the transfer container 14 that mixes with the sample to stabilize and/or preserve it. While a liquid buffer may be used in some cases, a liquid buffer is often undesirable because it may dilute the sample outside of specified tolerances. Toward this end, the transfer containers 14 may be equipped with a coating of solid material which does not affect the sample volume. Some possible examples include a preservative (e.g., azide), a pH adjusting agent (e.g., ascorbic acid), an amphipathic compound, and/or a viscosity reducing agent (e.g., amylase). In another embodiment, the disc of extraction surface 28 may comprise a fixed plastic surface that allows the sponge to be compressed. Another disk, suitable to contain a reagent (e.g., cellulose), having pre-dried reagents is also provided (e.g., the cellulose disk is soaked with reagent with water and subsequently freeze-dried) that could introduce stabilizing components without diluting the sample-therefore, no change in volume due to an additional liquid. The cellulose disc would typically be supported on extraction surface 28.

FIG. 8 illustrates various steps of methodology in accordance with one or more aspects of the present invention. As shown at 200, oral fluid (OF) is collected with the sponge of a collector. As shown at 202, the oral fluid is extracted into a transfer container. Depending on the embodiment, the collector may be discarded before subsequent steps, as indicated at 204. Sample portions are dispensed into laboratory containers, as indicated at 206. As indicated at 208, the lab containers are sealed, packaged, and transported to a testing facility.

While one or more preferred embodiments of the invention are described above, it should be appreciated by those skilled in the art that various modifications and variations can be made in the present invention without departing from the scope and spirit thereof.

What is claimed is:

1. An oral fluid collection assembly, comprising:
a collector having a stem carrying a collection media at a distal end thereof;
a transfer container having an extraction surface separating an interior of the transfer container into a first volume and a second volume;
the transfer container and the collector being connectable by latching features that cause engagement of the collection media and the extraction surface to produce an extracted sample; and
the transfer container having an outlet at its distal end so that the extracted sample can be dispensed from the transfer container.

2. An oral fluid collection assembly as set forth in claim 1, further comprising a plurality of laboratory containers configured to receive portions of the extracted sample from the transfer container.

3. An oral fluid collection assembly as set forth in claim 2, further comprising an adaptor to which the transfer container and at least two of the laboratory containers are connectable, the adaptor defining therethrough a flow passage from the outlet of the transfer container to respective inlets of the laboratory containers.

4. An oral fluid collection assembly as set forth in claim 1, wherein the collection media comprises a sponge mounted to a rigid mounting structure at the distal end of the stem.

5. An oral fluid collection assembly as set forth in claim 4, wherein the collector comprises a wicking strip providing a visual indication in the presence of the oral fluid.

6. An oral fluid collection assembly as set forth in claim 5, wherein the wicking strip is internal to the stem and is viewable through a transparent window in the stem.

7. An oral fluid collection assembly as set forth in claim 4, wherein the collector comprises a handling structure at a proximal end thereof.

8. An oral fluid collection assembly as set forth in claim 7, wherein the handling structure comprises a cap defining inner threads, the inner threads engaging outer threads on the transfer container.

9. An oral fluid collection assembly as set forth in claim 1, wherein the extraction surface comprises a disc situated at a fixed location in the transfer container, the disc defining at least one aperture for passage of the oral fluid.

10. An oral fluid collection device as set forth in claim 1, wherein the transfer container is squeezable.

11. An oral fluid collection assembly as set forth in claim 10, wherein the outlet of the transfer container comprises a dropper structure.

12. An oral fluid collection assembly as set forth in claim 1, wherein the transfer container has volume indicia to facilitate dispensing portions of the extracted sample.

13. An oral fluid collection assembly as set forth in claim 1, wherein the outlet of the transfer container is formed by a pierceable membrane.

14. An oral fluid collection assembly, comprising:
a collector having a stem carrying a rigid mounting structure at a distal end thereof to which a collection sponge is mounted, the collector further having a cap defining inner threads at a proximal end of the stem;
a transfer container having an extraction surface separating an interior of the transfer container into a first volume and a second volume, the extraction surface defining at least one aperture for passage of an extracted sample of the oral fluid;
the transfer container having outer threads at its proximal end engageable by the inner threads of the cap in a manner that causes engagement of the collection sponge and the extraction surface; and
the transfer container having an outlet at its distal end so that the extracted sample can be dispensed from the transfer container;
a plurality of laboratory containers configured to receive portions of the extracted sample from the transfer container.

15. An oral fluid collection assembly as set forth in claim 14, wherein the extraction surface comprises a disc situated at a fixed location in the transfer container.

16. An oral fluid collection assembly as set forth in claim 15, wherein the at least one aperture of the extraction disc comprises a plurality of radial slots.

17. An oral fluid collection assembly as set forth in claim 14, wherein the collector comprises a wicking strip providing a visual indication in the presence of the oral fluid.

18. An oral fluid collection assembly as set forth in claim 14, further comprising an adaptor to which the transfer container and at least two of the laboratory containers are connectable, the adaptor defining therethrough a flow passage from the outlet of the transfer container to respective inlets of the laboratory containers.

19. An oral fluid collection device as set forth in claim 1, wherein the transfer container is squeezable, and the outlet of the transfer container comprises a dropper structure.

20. A method of collecting a saliva sample from a user, the method comprising steps of:
obtaining an oral fluid collector having an elongate stem with a collection sponge located at its distal end;
collecting a sample with the collection sponge;
determining if the sample is sufficient;
if the sample is sufficient, compressing the sponge to produce an extracted sample into a squeezable transfer container having a dropper structure; and
squeezing the transfer container so as to dispense respective portions of the extracted sample into at least two separate laboratory containers.
